(19) United States
(12) Patent Application Publication
(10) Pub. No.: US 2018/0177986 A1
(43) Pub. Date: Jun. 28, 2018

(54) ADJUSTABLE BALLOON CATHETER DEVICE AND METHOD OF USE

(71) Applicant: Ashkan Farhadi, Irvine, CA (US)

(72) Inventor: Ashkan Farhadi, Irvine, CA (US)

(21) Appl. No.: 15/390,517

(22) Filed: Dec. 25, 2016

(51) Int. Cl. A61M 25/10; A61M 39/22

(52) U.S. Cl. CPC A61M 25/10182; A61M 25/1002; A61M 2025/1084; A61M 2039/229; A61M 2025/1068; A61M 39/22

(57) ABSTRACT

An adjustable catheter balloon is composed of an inflatable balloon, a flexible conduit and a handle. The inflatable balloon is attached to the distal end portion of the flexible conduit and the flexible conduit carries therewithin the core wire that moves independently within the flexible conduit and is moved using the handle. The balloon is inflated using a syringe that is connected to the handle which provides a relative movement of the core wire within the flexible conduit translating into changes in the length and contour of the balloon. This allows a diameter decrease of the adjustable catheter balloon and easier passage of the deflated balloon through a narrow working channel of an endoscopic device or a catheter. In addition we would have an increase in the diameter of the inflated balloon inside the body cavity by compressing the balloon longitudinally using the movement of the handle.

### FIELD OF THE INVENTION

This invention relates to balloon catheters typically used in diagnostic or therapeutic procedures. In particular, this invention relates to balloon catheters, which have to expand by inflation to relatively large dimensions while being passable through small channels.

### BACKGROUND OF THE INVENTION

Recently there are numerous medical interventions that have replaced major invasive surgeries by introducing endoscopic, catheter based or laparoscopic procedures. In these procedures, there is no direct access to the site by the operator and instead the operator uses tools to access the inaccessible site in the body through small passage channels provided by endoscope, laparoscope or catheter. Catheter balloons are one of the instrumental tools in most of these procedures, which are typically used for dilatation, or obstruction of a duct or body lumen. Balloon catheter is typically composed of a thin catheter that has a proximal end for manual deployment and attachment to syringe for inflation and deflation and a distal end that is attached to an expandable balloon. Balloons are deflated during passage through the provided passage channel and when in the body cavity are inflated using air or fluid using the catheter, which is in fluid communication with the balloon.

The size of the inflated balloon is limited to some factors and cannot exceed a certain limit due to the elastic property of the balloon material. In addition adding to the size of the inflated balloon can result in enlargement of the size of the deflated balloon, which would be limited, by the size of the passage channel. Not to mention that deflated folded balloon material could create significant friction while traversing through the passage channel.

### SUMMARY OF THE INVENTION

The intention of this invention is to describe a new catheter balloon that allows passage of the deflated catheter balloon from a smaller channel and create a larger inflated catheter balloon in the body cavity. To achieve this objective, the current invention introduces an inflatable balloon catheter composed of a flexible conduit, an inflatable balloon, a handle and a core wire. The inflatable balloon is attached and inflated by the flexible conduit. The flexible conduit carries therewithin the core wire that moves independently of the flexible conduit. The inflatable balloon is attached to the distal end portion of the flexible conduit on its proximal end and to the distal end of the core wire on its distal end. The relative movement of the core wire within the flexible conduit permits modulation of the contour of the balloon. A handle and an inflation port are provided at the proximal end portion of the flexible conduit. The inflation port on the handle is in fluid communication with the inflatable balloon through the flexible conduit and can be attached to a syringe for inflation of deflation of the balloon. The handle is also provided with two moving parts that are attached to the proximal end portion of the flexible conduit and the proximal end portion of the core wire and can be grasped by operator's fingers. The movement in the handle is translated to a relative movement of the flexible conduit and the core wire at their distal ends, which in turn changes the contour of the inflatable balloon. Stretching the balloon when it is deflated results in an increase in the balloon length and a decrease in the effective diameter of balloon width, which facilitates the passage of the balloon through the provided channel. Compressing the balloon when it is inflated on the other hand, results in augmentation of the effective diameter of the inflated balloon, which improves the intended balloon function (larger size).

### BRIEF DESCRIPTION OF DRAWINGS

FIG. 1 is a schematic illustration of the device with an attached syringe.

FIG. 2 is a top view of the device handle in FIG. 1 without attached syringe in the neutral position.

FIG. 3 is a top view of the device handle in FIG. 1 without attached syringe in the stretched position.

FIG. 4 is a top view of the device handle in FIG. 1 without attached syringe in the compressed position.

FIG. 5 is a limited longitudinal sectional view along plane 1-1 of FIG. 1.

FIG. 6A is a limited longitudinal sectional view of the device shown in FIG. 1 taken along plane 2-2 of FIG. 1 while inflatable balloon is deflated in its neutral position and the inflatable balloon is not being longutidinally stretched.

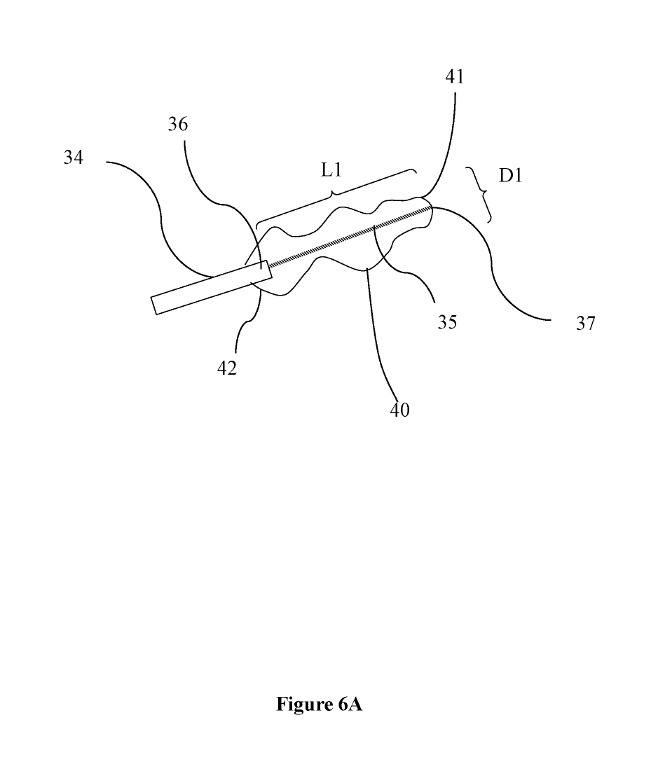

FIG. 6B is a sectional view of the device shown in FIG. 1 taken along plane 2-2 while inflatable balloon is deflated and the inflatable balloon is being longutidinally stretched.

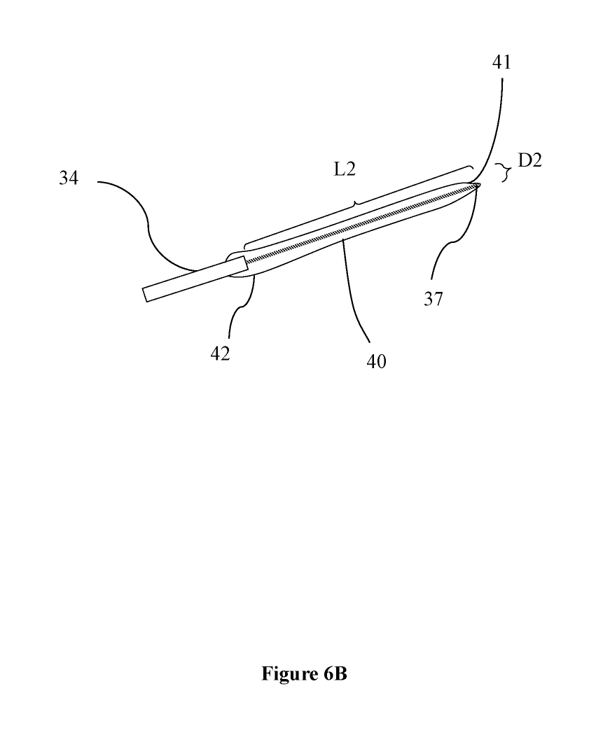

FIG. 6C is a sectional view of the device shown in FIG. 1 taken along plane 2-2 while inflatable balloon is inflated in its neutral position and the inflatable balloon is not being longutidinally compressed.

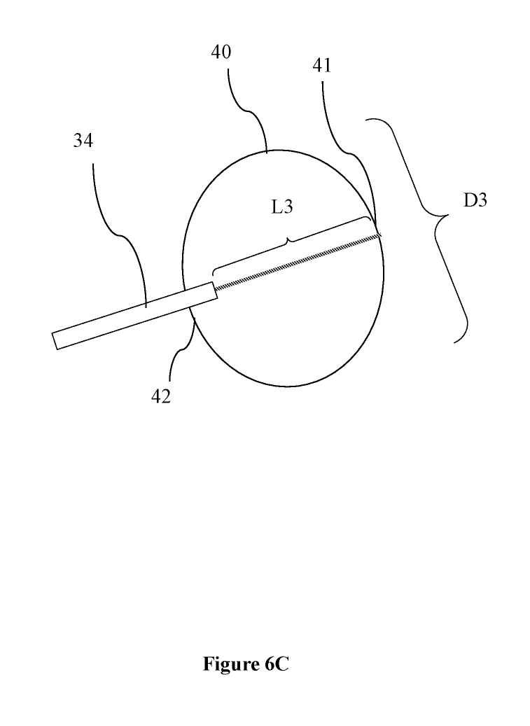

FIG. 6D is a sectional view of the device shown in FIG. 1 taken along plane 2-2 while inflatable balloon is inflated and the inflatable balloon is being longutidinally compressed. The length of the inflated balloon in the compressed form L4 is shorter than length of the inflated balloon in the neutral form L3 while the diameter of the inflated balloon in the compressed form D4 is larger than the diameter of the inflated balloon in the neutral form D3.

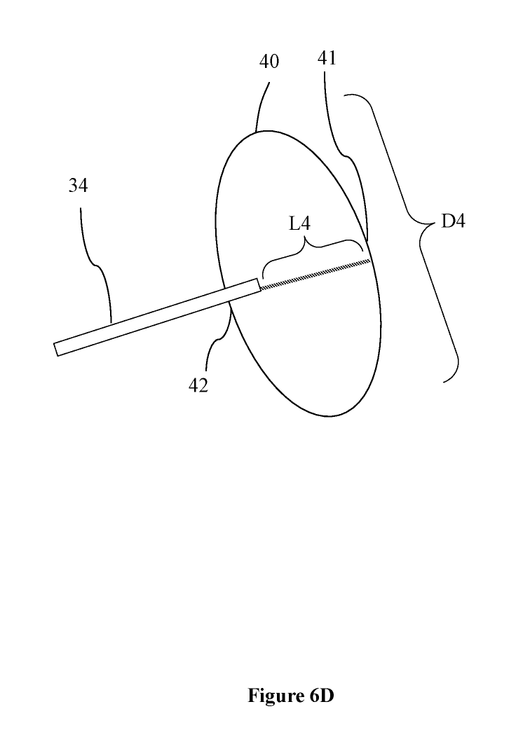

FIG. 7A is a sectional view of the device shown in FIG. 1 taken along plane 3-3 while inflatable balloon is deflated and the inflatable balloon is not being longutidinally stretched. The diameter of the deflated balloon in the neutral form D1 is shown.

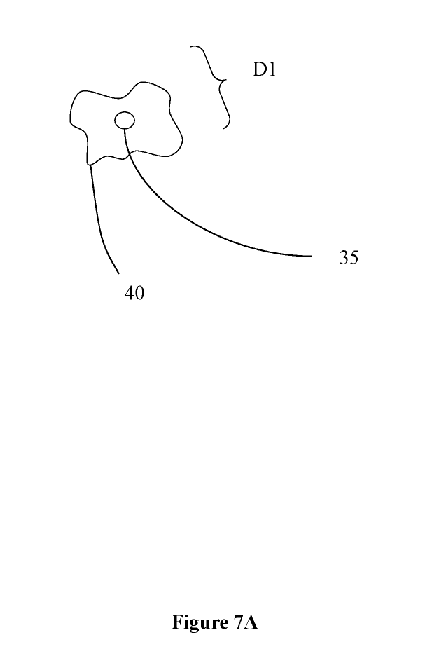

FIG. 7B is a sectional view of the device shown in FIG. 1 taken along plane 3-3 while inflatable balloon is deflated and the inflatable balloon is being longutidinally stretched. The diameter of the deflated balloon in the stretched form D2 is smaller than the diameter of the deflated balloon in the neutral form D1.

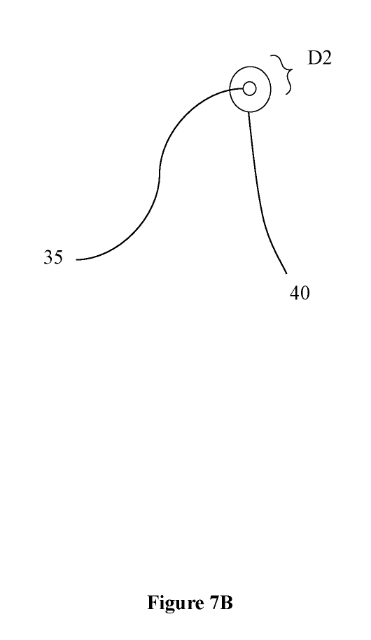

FIG. 7C is a sectional view of the device shown in FIG. 1 taken along plane 3-3 while inflatable balloon is inflated and the inflatable balloon is not being longutidinally compressed. The diameter of the inflated balloon in the neutral form D3 is shown.

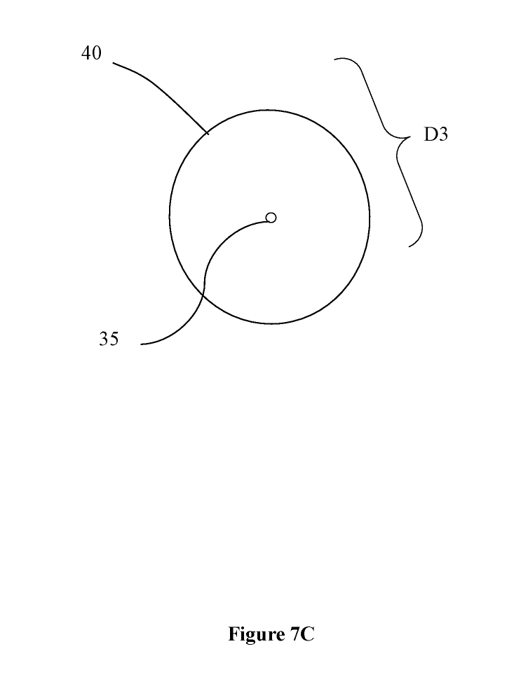

FIG. 7D is a sectional view of the device shown in FIG. 1 taken along plane 3-3 while inflatable balloon is inflated and the inflatable balloon is being longutidinally compressed. The diameter of the inflated balloon in the compressed form D4 is larger than the diameter of the inflated balloon in the neutral form D3.

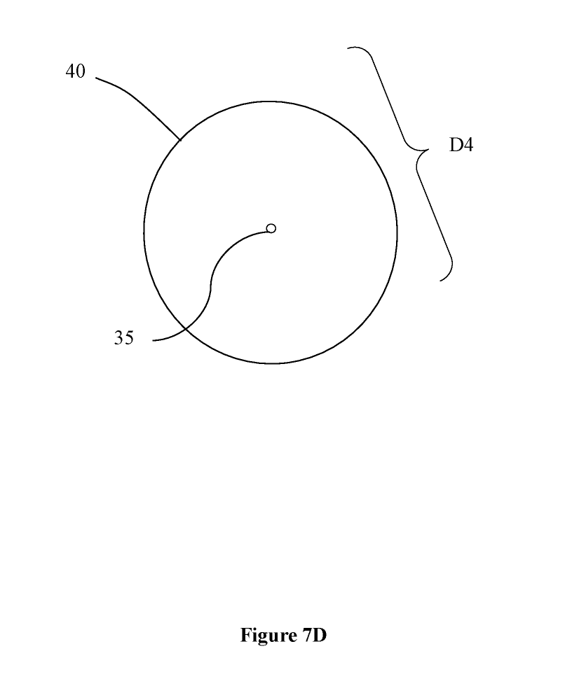

FIG. 8 is a flowchart regarding the method of use, according to an embodiment of the present invention.

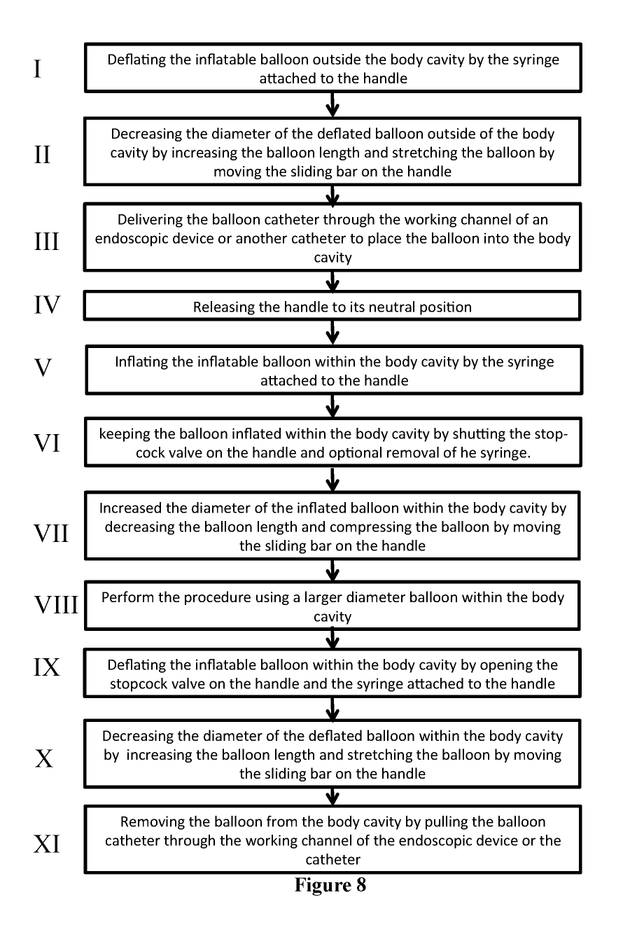

### DETAILED DESCRIPTION OF DRAWINGS

Before explaining some aspects of embodiment of the present invention in detail, it is to be understood that the present invention is not limited in its application to the details of arrangements of the components set forth in the following description. As will be appreciated by those skilled in the arts, the present invention is capable of other embodiments and of being practiced and carried out in various ways. Also, it is to be understood that the phraseology and terminology employed herein are for the purpose of description and should not be regarded as limiting. It is also to be understood that where ranges are provided for various aspects of the invention and for examples, they are approximate ranges and are not to be limiting except where noted otherwise.

Unless defined otherwise, all technical and scientific terms used herein have the same meaning as commonly understood by one of ordinary skill in the art to which this invention belongs. Moreover, the singular forms “a”, “an”, and “the” include plural referents unless the context clearly dictates otherwise. Additionally, while the word examination is used, it is meant to include also diagnosis, treatment or therapy as may be the case.

According to certain embodiments of the present invention, a balloon catheter 10 may include:

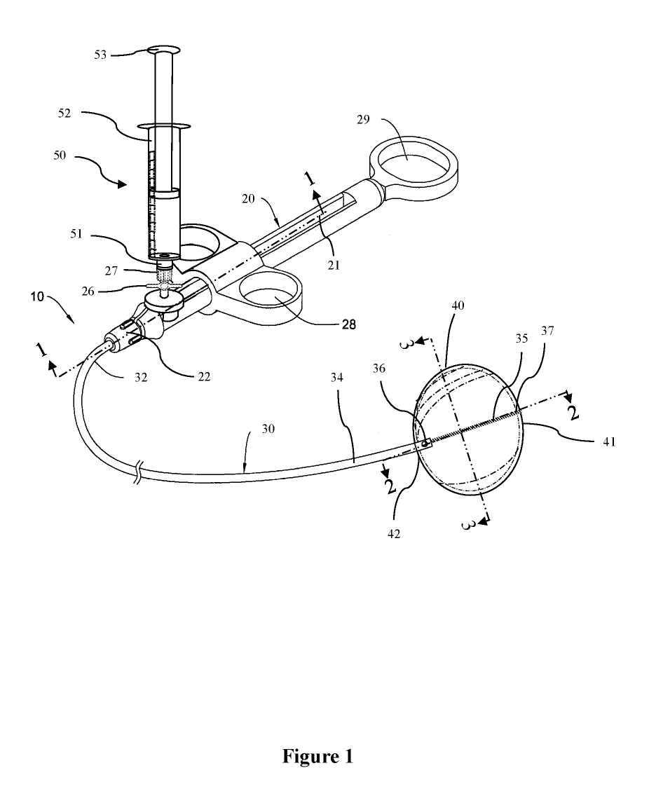

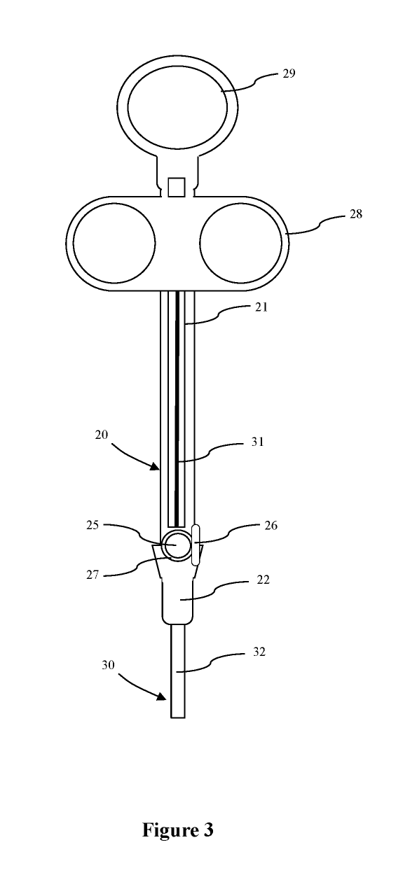

As it is depicted in FIG. 1, the adjustable balloon catheter 10 is comprised of a handle 20 and inflatable balloon 40 connected by a longitudinally elongated catheter or flexible conduit 30 and a removably attached syringe 50. The flexible conduit 30 has a proximal end portion 32 connected to the handle 20 and a distal end portion 34 connected to the balloon 40. FIG. 3 depicts the proximal end portion 32 of the flexible conduit 30 is affixed to a hub 22 at the end portion of the handle 20 and within the handle 20 is in flow communication with a port 23 which communicate with port 25 that can be shut or opened using a stopcock valve 26 and terminates in a lure connection 27. The lure connection 27 can be connected to a tip 51 of the syringe 50. The stopcock valve 26 may be closed to prevent air from passing through the port 25.

The distal end portion 34 of the flexible conduit 30 is affixed to a proximal portion of the inflatable balloon 40. A port 36 in conduit 30 situated within the inflatable balloon 40 allows fluid communication of air for inflation or deflation of the inflatable balloon 40 through flexible conduit 30, based on air pressure from the syringe 50.

With further reference to FIG. 1, the flexible conduit 30 also carries therewithin a core wire 31 that is movable slideably within and independently of the flexible conduit 30. The relative movement of the core wire 31 within the flexible conduit 30 creates the movement of the first end portion 35 of the core wire 31 relative to the distal end portion 34 of the flexible conduit 30, such that the wire enters the balloon 40. FIG. 3 depicts the core wire 31 enters the handle 20 through the hub 22, passes through the port 23 and a sealing washer 24 and is affixed attaches to a terminus 39 in the center of the sliding bar 28 of the handle 20. Movement of the sliding bar 28 within the groove 21 of the handle 20 moves the core wire 31 within and relative to the flexible conduit 30.

The first end portion 35 of the core wire 31 extends beyond the distal end portion 34 of the flexible conduit 30 and moves independently of the conduit 30. The tip 37 of the core wire 31 is affixed to the far end 41 of the inflatable balloon 40 and the distal end portion 34 of the conduit 30 is attached to the opening 42 of the inflatable balloon 40. Movement of the end portion 35 of the core wire 31 within the flexible conduit 30 changes the distance between the distal end portion 41 and proximal end portion 42 of the balloon 40 and this results in altering the contour of the inflatable balloon 40.

As shown in FIG. 1, the inflatable balloon 40 is made from an elastomeric material and is attached at its far end 41 to the tip 37 of the core wire 31 and at its opening 42 to the distal end portion 34 of the flexible conduit 30. The inflatable balloon 40 is inflated or deflated through distal port 36 at the distal end portion 34 of the conduit 30 that is in flow communication with a port 23 within hub 22 which, communicate with port 25 that can be shut or opened using a stopcock valve 26 and terminates in a lure connection 27. The Lure connection 27 can be removaly connected to the tip 51 of the syringe 50. The stopcock valve 26 may be closed to prevent air from passing through the port 25. The port 23 is connected to the conduit 30 at it distal end portion and is supplied with a washer 24 at its proximal end that encircles the wire 31 and allows movement of wire 31 therewithin while maintaining the air pressure in the port 23. The length of the inflatable balloon 40 is adjusted by movement of the core wire 31 within the flexible conduit 30.

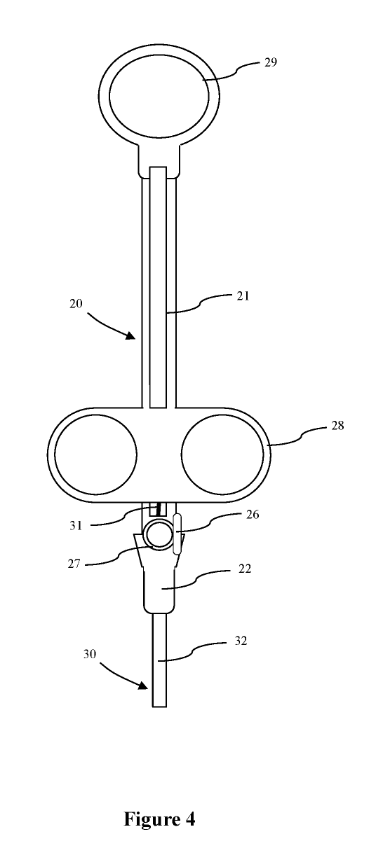

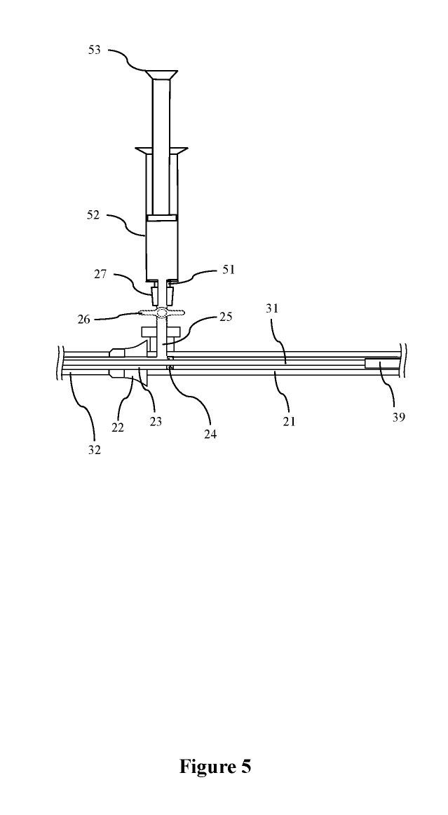

As shown in FIGS. 4 and 5, the inflatable balloon 40 length can be adjusted by movement of the core wire 31 within the conduit 30. This movement changes the contour of the inflatable balloon 40.

With reference to FIGS. 4A and 4B, the length of the balloon L1 in FIG. 4A can be increased to L2 FIG. 4B while the balloon 40 is deflated using the handle 20 and movement of wire 31 within the conduit 30. This changes in the length stretches the deflated balloon 40 and while the length is longer, the diameter of the balloon 40 D2 in FIG. 4B is smaller than D1 in FIG. 4A.

With reference to FIGS. 4C and 4D, the length of the balloon L3 in FIG. 4C can be decreased to L4 FIG. 4B while the balloon is inflated using the handle 20 and movement of wire 31 within the conduit 30. This changes in the length compresses the inflated balloon 40 and while the length is shorted, the diameter of the balloon D3 in FIG. 4C is larger than D4 in FIG. 4D.

FIG. 5A is a sectional view of the balloon 40 and corresponds to FIG. 4A, taken along plane 3-3 while balloon 40 is deflated. FIG. 5B is a sectional view of the balloon 40 and corresponds to FIG. 4B, taken along plane 3-3 while balloon 40 is deflated. In FIG. 5A the inflatable balloon 40 is not being longitudinally stretched. The diameter of the balloon 40 is D1 in FIG. 5A that can be decreased to D2 FIG. 5B due to the action of the handle 20 and movement of wire 31 within the conduit 30 when the length of balloon 40 is increased.

FIG. 5C is a sectional view of the balloon 40 and corresponds to FIG. 4C, taken along plane 3-3 while balloon 40 is inflated. FIG. 5D is a sectional view of the balloon 40 and corresponds to FIG. 4D, taken along plane 3-3 while balloon 40 is inflated. In FIG. 5C the inflatable balloon 40 is not being longitudinally compressed. The diameter of the balloon 40 is D3 in FIG. 5C that can be increased to D4 in FIG. 5D due to the action of the handle 20 and movement of wire 31 within the conduit 30 when the length of balloon 40 is decreased.

As shown in FIGS. 1, 2 and 5, handle 20 is affixed to the proximal end portion 32 of the flexible conduit 30 at hub 22. The core wire proximal end portion is carried within the flexible conduit 30 and is attached to the terminus 39 of the sliding bar 28 in the handle 20. The sliding bar 28 moves back and forth in a linear motion along the groove 21 within the handle 20 and moves with it the core wire 31, which moves within the flexible conduit 30. The sliding bar 28 is supplied with two finger rings adapted to receive index and middle finger of operator. The proximal part of the handle 20 is supplied with a plunger ring 29 adapted to receive the operator's thumb.

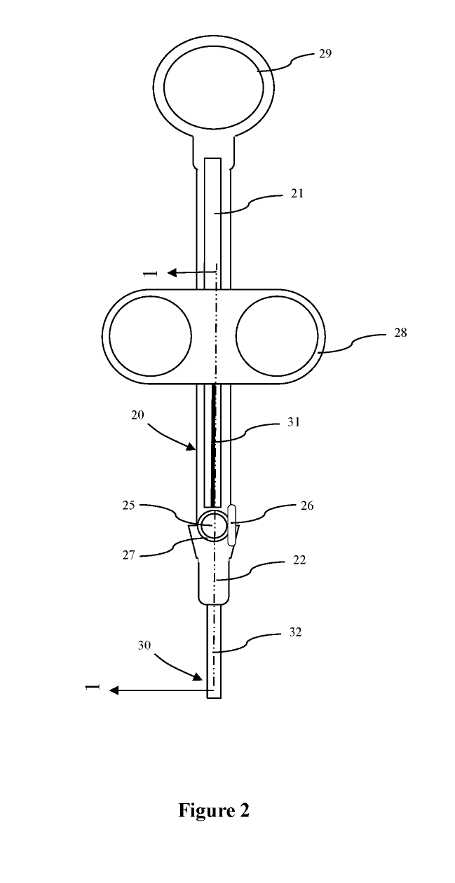

As shown in FIGS. 2, 3 and 4, the sliding bar 28 of the handle 20 moves back and forth in a linear motion along the groove 21 within the handle 20 and moves with it the core wire 31, which moves within the flexible conduit 30. The sliding bar 28 is supplied with two finger rings adapted to receive index and middle finger of operator. The proximal part of the handle 20 is supplied with a plunger ring 29 adapted to receive the operator's thumb. FIG. 2 depicts the sliding bar at its neutral position. When the sliding bar 28 is moved by pulling it toward the plunger ring 29 as depicted in FIG. 3, the wire 31 is pulled and this results in decreasing the length of the balloon 40. When the sliding bar 28 is moved by pushing away from the plunger ring 29 as depicted in FIG. 4, the wire 31 is pushed in the conduit 30 and this results in increasing the length of the balloon 40.

In addition, the handle 20 is supplied with a lure connection 27 to removably attach the syringe 50 that is in fluid communication with flexible conduit 30 through port 25 and 23. The proximal end portion of port 23 of the handle 20 is supplied with a washer 24 that hydraulically seals the port 23 of the handle 20 when the core wire 31 moves through this portion back and forth through the washer 24. This configuration allows for inflation or deflation of the balloon 40 using syringe 50. The port 25 is also supplied with a stopcock valve 26, which may be selectively closed to seal air within the balloon 40.

Method of use: Adjustable balloon catheter 10 is used for passage through a channel of the device or a catheter to be deployed in the body cavity. The advantage of the adjustable balloon catheter 10 over regular catheter balloon is in decreasing the diameter of the balloon catheter during the passage from a narrow working channel of a device or a catheter while deflated and increasing the diameter of the inflated balloon inside the body cavity while inflated.

To this end, step I-IV consists of the passage of the deflated balloon 40 of adjustable balloon catheter 10 through an endoscopic device working channel or a catheter into a body cavity. This step consist of:

Step I: Deflating the inflatable balloon 40 outside the body cavity by pulling the plunger 53 of the syringe 50 attached to the handle 20.

Step II: The handle is grasped by three fingers and the sliding bar 28 is moved toward the plunger ring 29. The movement of the sliding bar 28 relative to plunger ring 29 moves the wire 31 relative to conduit 30 which result in stretching the deflated balloon 40 longitudinally from its neutral deflated length L1 to stretched deflated length L2 and in turn decreases the diameter of the deflated balloon 40 from its neutral deflated diameter D1 to stretched deflated diameter D2 outside of the body cavity. This allows a better passage of the adjustable balloon catheter 10 through narrow working channel of an endoscopic device or a catheter into a body cavity.

Step III: The tip 37 of the wire 31 is entered into a working channel of an endoscopic device or a catheter to deliver the deflated balloon 40 into the body cavity by pushing the catheter balloon 10 into the working channel of an endoscopic device or a catheter.

Step IV: The handle is released to its neutral position.

Step V-VIII consists of the performing procedures with the inflated balloon within the body cavity after passage of the deflated balloon 40 of adjustable balloon catheter 10 through the endoscopic device channel or the catheter into a body cavity. This step consist of:

Step V: Inflating the inflatable balloon 40 within the body cavity by depressing the plunger 53 into the cylinder 52 of the syringe 50 that send air into port 25 when the stopcock 26 is in open position. The air passes through port 25 into port 23 into lumen of the conduit 30 into port 36 at the end portion 34 of the conduit 20 and enters the balloon 40.

Step VI: Keeping the balloon 40 inflated within the body cavity by shutting the stopcock valve 26 on the handle 20. The washer 24 creates a hydraulic seal around wire 31 when it moves within the port 23 for adjusting the length and diameter of the balloon 40. This maintains the air pressure created within the balloon 40 by syringe 50. After inflation of the balloon 40 using syringe 50 and closing of the stopcock 26 the syringe 50 can optionally be removed by disconnecting lure 27 of the handle 20 from the tip 51 of the syringe 50.

Step VII: The handle is grasped by three fingers and the sliding bar 28 is moved away from the plunger ring 29. The movement of the sliding bar 28 relative to plunger ring 29 moves the wire 31 relative to conduit 30 which result in compressing the inflated balloon 40 longitudinally from its neutral inflated length L3 to compressed inflated length L4 and in turn increases the diameter of the inflated balloon 40 from neutral inflated diameter D3 to compressed inflated diameter D4 within the body cavity. This results in increased the diameter of the inflated balloon 40, which creates a larger balloon diameter of the adjustable balloon catheter 10, compared to non-compressed balloon within the body cavity.

Step VIII: Performing the procedure using a larger diameter balloon within the body cavity.

Step IX-XI consists of the removal of the balloon catheter 10 through the endoscopic device channel or the catheter after deflating balloon 40 from the body cavity. This step consist of:

Step IX: Reconnecting the syringe 50 (if it was removed before) to the handle 20 using lure 27 of the handle 20 and the tip 51 of the syringe 50. Opening the stopcock 26 valve on the handle 20 and deflating the inflatable balloon 40 inside the body cavity by pulling the plunger 53 of the syringe 50 attached to the handle 20.

Step X: The handle is grasped by three fingers and the sliding bar 28 is moved toward the plunger ring 29. The movement of the sliding bar 28 relative to plunger ring 29 moves the wire 31 relative to conduit 30 which result in stretching the deflated balloon 40 longitudinally from its neutral deflated length L1 to stretched deflated length L2 and in turn decreases the diameter of the deflated balloon 40 from its neutral deflated diameter D1 to stretched deflated diameter D2 within the body cavity.

Step XI: Removing the deflated balloon 40 from the body cavity by pulling the balloon catheter 10 through the endoscopic device working channel or the catheter.

I claim:

1. An adjustable balloon catheter device for diagnostic and therapeutic intervention comprising:
a handle, a flexible conduit connecting the handle to an inflatable balloon, a syringe for inflating the inflatable balloon and a core wire slidably disposed within the flexible conduit for changing the length of the balloon and wherein the inflatable balloon is operated between a first neutral position to second stretched or third compressed position using sliding bar in the handle adjusting the balloon length, diameter and contour.

2. The device of claim 1, further comprising:
a. a handle comprising:
i. a groove disposed along a length of the handle;
ii. a sliding bar slidably engaged to the groove;
iii. a hub engaged to an end of the handle; and
iv. a lure connection, wherein the lure connection is releasably engaged with a syringe;
b. a flexible conduit having a proximal end portion and a distal end portion, wherein the proximal end portion is in communication with the hub, wherein the distal end portion is in communication with the balloon;
c. an inflatable balloon positioned at the distal end portion of the flexible conduit;
d. a core wire slidably disposed within the flexible conduit, the core wire having a first end portion and second end portion, wherein the first end portion extends to a far end of the inflatable balloon and terminates at a core wire tip, and the second end portion extends and terminates at the sliding bar of the handle.

3. The device of claim 1, further comprising:
a series of ports and conduit for passage of air from the syringe into the inflatable balloon comprising of a first and second port and a flexible conduit.

4. The device of claim 1, further comprising:
the first port of claim 3, further comprising of two ends, wherein one end is in fluid communication with proximal end portion of the flexible conduit through the hub and the other end of the first port is in fluid communication with the second port, wherein the first port sealably receives the core wire.

5. The device of claim 1, further comprising:
the second port of claim 3, further comprising of two ends, wherein one end is in fluid communication with the first port in a right angle connection and the other end is in fluid communication with the syringe that can removably attached to the end of the second port through a lure, wherein the second port receives a stopcock valve that can control the passage of air within the second port.

6. The device of claim 1, wherein the wire is adapted to modify a balloon length, a diameter, and a contour, wherein the length, diameter and contour are adjusted by the slidably engaged sliding bar.

7. The device of claim 1, wherein a stopcock valve controls the passage of air into and out of the inflating balloon.

8. The device of claim 1, having a sealing washer, wherein the sealing washer maintains a pressure in the balloon while the size and diameter of the balloon can be adjusted using the wire.

9. The device of claim 1, wherein the inflatable balloon length and inflating air pressure are adjustable independently.

10. The device of claim 1, wherein the inflated balloon diameter and contour can be modified by either the amount of air pressure inflated within the balloon and or altering the length of the balloon independently.

11. The device of claim 1, wherein the deflated balloon diameter and contour can be modified by altering the length of the balloon.

12. The device of claim 1, wherein the alteration of the deflated balloon contour by stretching the deflated balloon reduces the diameter of the deflated balloon that facilitate the passage of the deflated balloon through a working endoscopic channel or a catheter into the body cavity.

13. The device of claim 1, wherein the diameter of the balloon catheter can be reduced beyond full deflation of the balloon.

14. The device of claim 1, wherein the diameter of the balloon catheter can be increased without need for further inflation of the balloon.

15. The device of claim 1, wherein the pressure within the balloon can be increased without need for further inflation of the balloon.

16. The device of claim 1, wherein the contour of the balloon can be altered without alteration in the amount of balloon inflation.

17. The device of claim 1, wherein the alteration of the inflated balloon contour by compressing the inflated balloon increases the diameter of the inflated balloon that make it possible to acquire a larger diameter balloon within the body cavity during the procedures.

18. An adjustable balloon catheter device for diagnostic and therapeutic intervention comprising:
a handle, a flexible conduit connecting the handle to an inflatable balloon, a syringe for inflating the balloon and a core wire slidably disposed within the flexible conduit for changing the length of the balloon and wherein the inflatable balloon is operated in deflated position between a first neutral position to second stretched position using sliding bar in the handle adjusting the balloon length, diameter and contour and wherein the diameter of the deflated balloon can be reduced by stretching the deflated balloon.

19. An adjustable balloon catheter device for diagnostic and therapeutic intervention comprising:
a handle, a flexible conduit connecting the handle to an inflatable balloon, a syringe for inflating the balloon and a core wire slidably disposed within the flexible conduit for changing the length of the balloon and wherein the inflatable balloon is operated in inflated position between a first neutral position to second compressed position using sliding bar in the handle adjusting the balloon length, diameter and contour and wherein the diameter of the inflated balloon can be increased by compressing the inflated balloon.

20. An adjustable balloon catheter device method of use comprising:
I. deflating the inflatable balloon outside the body cavity by a syringe on the handle;
II. stretching the deflated balloon by moving sliding bar toward the plunger ring on the handle, wherein increasing the length of the inflatable balloon decreases the diameter of the balloon;
III. delivering the balloon catheter into a body cavity through endoscopic working channel or catheter;
IV. releasing the sliding bar to its neutral position after passage of the balloon into the body cavity;
V. inflating the inflatable balloon by the syringe on the handle;
VI. maintaining the inflation by closing the stopcock valve;
VII. decreasing the length of the inflated balloon by moving the sliding bar away the plunger ring on the handle, wherein decreasing the length of the balloon increases the diameter of the balloon;
VIII. performing the procedure using a larger diameter balloon within the body cavity;
IX. deflating the inflated balloon by opening the stopcock valve and the syringe on the handle;
X. stretching the deflated balloon by moving sliding bar toward the plunger ring on the handle, wherein increasing the length of the inflatable balloon decreases the diameter of the balloon;
XI. removing the balloon catheter from a body cavity, endoscopic working channel or catheter.
